(19) United States
(12) Patent Application Publication
(10) Pub. No.: US 2002/0120246 A1
(43) Pub. Date: Aug. 29, 2002

(54) Bag-type tampon containing compressed fibrous material

(76) Inventor: Herve Buzot, North Brunswick, NJ (US)

Correspondence Address: Philip S. Johnson, Esq., Johnson & Johnson, One Johnson & Johnson Plaza, New Brunswick, NJ, 08933-7003, US

(21) Appl. No.: 09/741,718

(22) Filed: Dec. 20, 2000

(51) Int. Cl. A61F 13/15

(52) U.S. Cl. 604/385.17; 28/121

(57) ABSTRACT

An absorbent article having a nonwoven pouch and tablets of absorbent material, said absorbent material comprising compressed fiber having a predetermined density, said predetermined density within 20% of the fiber's maximum volume.

### FIELD OF THE INVENTION

The invention relates to novel absorbent articles, such as catamenial tampons and pads. More particularly, the present invention relates to bag-type tampons containing compressed fibrous materials.

### BACKGROUND OF THE INVENTION

Commercial catamenial tampons are often comprised of an absorbent body of moderately compressed fibers, and these tampons are generally in the shape of a cylinder or a bullet. Examples of such tampons are the commercially available o.b.® tampons. Such tampons have dimensions varying from 45 mm to 50 mm in length and 11 mm to 17 mm in diameter. These tampons are generally described in Friese et al., EP 422 660, Friese, U.S. Pat. No. 4,816,100, and Nguyen et al., U.S. Pat. No. 5,750,446. Commercial tampons generally have a density of about 0.4 to 0.5 g/cc.

A second type is a tampon that is more prevalent in the patent art than it is commercially available has multiple pieces of absorbent material encased within a porous overwrap. This is commonly known as a bag-type tampon. The bag-type tampon provides certain advantages over the first tampon type. They may have greater absorbent capacity than commercial tampons, may have more bulk for containment of fluids, and the particulate absorbent provides a large amount of surface area.

For example, Schaefer, U.S. Pat. No. 3,815,601, discloses a tampon wherein the absorbent body is an aggregate of separate pieces of low modulus, resilient, absorbent foam encased within a fluid permeable overwrap. The aggregate may also include an ancillary absorbent material to hold liquids within the absorbent body after they have been absorbed and thus reduce squeeze-out when a liquid loaded tampon is compressed.

Reeves et al, U.S. Pat. No. 4,278,088, discloses a bag-type tampon purporting to be an improvement over Schaefer. This tampon has discrete pieces of a compressed dry shape-retaining absorbent rigid paper-like matt. The pieces are formed by cutting a compressed paper-like sheet or matt into small, discrete pieces, generally about 1/16″ to 1/2″ in length and width or strips of about 1/4″ to 1/8″ in width and about 1″ to 3″ in length. These pieces may be loosely dispersed or rolled in a fluid-permeable bag. The matt is compressed to a thickness of about 0.5 to 0.1 times the original thickness of uncompressed fibrous material, and the lower level of compression is indicated as being better. The matt can have as its major component compressible cellulosic fibers. Reeves purports that its dry tampon does not expand after insertion, nor does it have the bulk associated with Schaefer, U.S. Pat. No. 3,815,601.

While Reeves is an interesting evolution of the bag-type tampon of Schaefer, the product described therein fails to take advantage of the benefits available to the bag-type tampon technology.

Therefore, what is needed is a bag-type tampon that is capable of absorbing adequate amounts of bodily liquids while comfortably conforming to the user's body and that can expand to effectively fill the vagina during use, thereby helping to reduce by-pass leakage.

### SUMMARY OF THE INVENTION

The present invention relates to an absorbent article having a fluid-permeable overwrap containing a plurality of tablets of compressed, fibrous, absorbent material. The absorbent material has a bulk density within about 20% of said absorbent material's maximum volume capacity.

In an alternative embodiment, the absorbent article has a liquid-permeable bag containing a plurality of tablets of compressed, fibrous, absorbent material. The tablets are compressed to a bulk density of at least about 0.5 g/cm³.

Finally, the invention also relates to a method of making an absorbent article. The method includes comprising the steps mixing fibrous, absorbent material, forming the mixed absorbent material into compressed tablets, placing the compressed tablets into an overwrap, and sealing the overwrap.

### BRIEF DESCRIPTION OF THE DRAWINGS

FIG. 1 shows a partially cut-away side elevation of an absorbent article according to one embodiment of the present invention.

FIG. 1A shows an enlarged view of several tablets contained within the absorbent article of FIG. 1.

FIG. 2 shows a partially cut-away side elevation of a tampon according to a second embodiment of the present invention.

FIG. 2A shows an enlarged view of several tablets intermixed with loose fibers contained within the tampon of FIG. 2.

FIG. 3 shows a partially cut-away side elevation of a tampon contained within a tampon applicator according to a third embodiment of the present invention.

FIG. 4 shows an exploded view of the tablets forming the tampon of FIG. 3.

### DETAILED DESCRIPTION OF THE INVENTION

As used herein, the term “absorbent article” generally refers to articles used to absorb and contain body exudates, and more specifically, to articles that are placed against, in proximity to, or inside the body of the wearer to absorb and contain such body exudates. The term includes, without limitation, diapers, catamenial pads, tampons, sanitary napkins, incontinence articles, training pants, and the like, as well as wipes, bandages, and wound dressings.

As used herein, the term “bulk density” generally refers to the density of a mass of material, such as fibers, including interfiber volume. Thus, the bulk density of a mass of fibers will be less than the density of the individual fiber due to the inclusion of these voids.

An absorbent article comprising a fluid-permeable bag and a plurality of tablets of compressed, fibrous, absorbent material is desirable, as this configuration can provide good expansion, high fluid retention, high fluid volume capacity, and an amorphous structure that can conform to body contours as used.

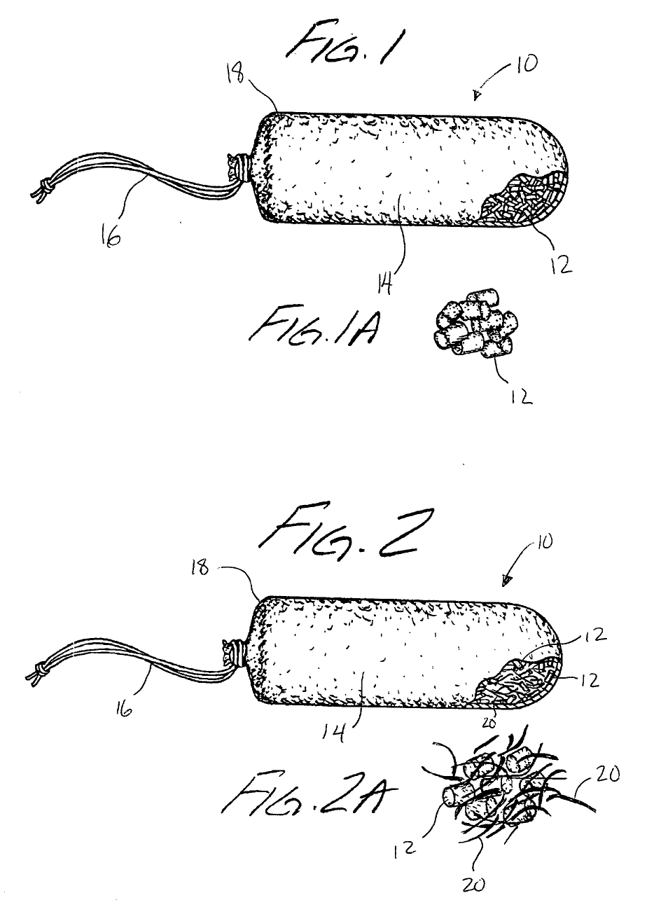

In a first embodiment shown in FIGS. 1 and 1A, a bag-type tampon 10 is prepared using small cylindrical tablets 12 of absorbent, compressed fibers with higher density as described below. These tablets 12 are contained within an overwrap 14, and there is a withdrawal 16 string attached to the withdrawal end 18 of the tampon 10.

FIGS. 2 and 2A show a second embodiment in which loose fibers 20 (or other loose materials) are combined with the compressed tablets 12 of absorbent, fibrous material that are contained by the overwrap 14. The bulkiness of the bag-type tampon 10 is improved by the addition of loose fibers 20 and the absorbency is provided by the highly compressed tablets 12. The surface area and, hence, absorbency are both increased in this embodiment.

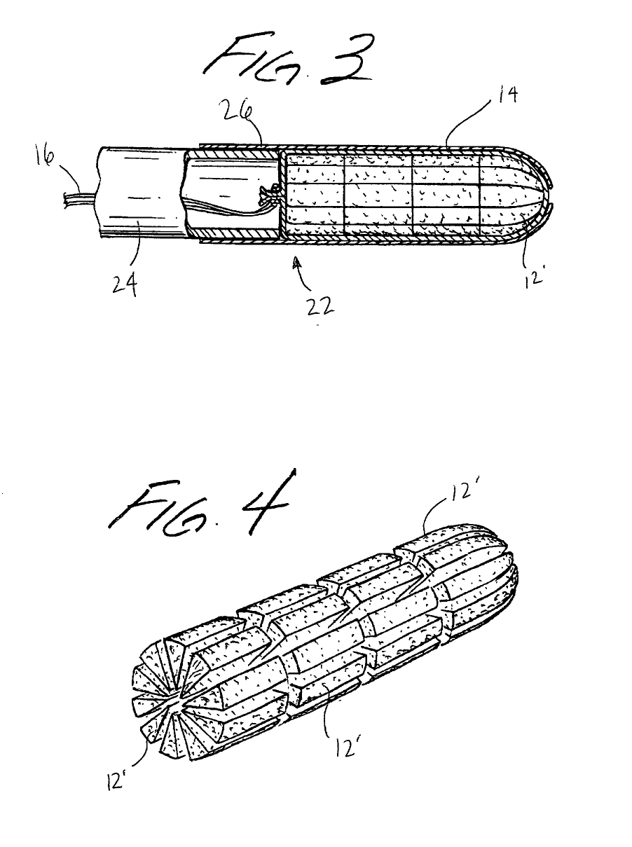

FIGS. 3 and 4 illustrate a third embodiment. A bag-type tampon 10 is prepared in which the compressed tablets 12′ are assembled as segments of a tampon (bullet) shape, and they are enclosed within a non-woven cover 14. The bag-type tampon 10 is placed inside applicator 22 having a plunger 24 to expel the tampon 10 from within a barrel 26. Once inserted into cavity, the tablets 12′ can disassemble to create loose structure with large open areas. The surface area available for absorption is again increased. This allows for fast fluid uptake and transport.

Steiger et al., “Absorption of Liquid by Compressed Fiber Systems”, Textile Research Journal, Vol. 42, No. 8, pp. 443-449 (1972), describes how the absorbency of fiber systems, particularly at various bulk densities, perform. This article teaches that the weight capacity (“C_{w}”) of a fibrous system decreases as bulk density increases (a substantially linear relationship). The article also discloses a volume capacity (“Cᵥ”) determined on the basis of capacity per unit of original dry-bulk volume of fibers. This volume capacity can be determined by multiplying the original C_{w} by the original bulk density (“D”), or Cᵥ=C_{w}×D. An interesting feature of volume capacity is that it exhibits a maximum value at a bulk density much higher than conventional absorbent structures possess.

While not wishing to be held to this theory, it appears that, as the bulk density of the fibrous mass increases, interfiber capillarity increases to improve fluid holding capacity of the mass and the proportion of fibers that become plastically deformed (or broken) also increases. As density increases, more fibers start becoming plastically deformed and broken, and the interfiber capillaries become shorter and less efficient. Thus, at the Cᵥ, the benefit of added absorbent material balances the damage done to the efficiency of the fibrous mass. Past the Cᵥ, the benefit of incorporating more absorbent fibers in a defined plug volume is outweighed by having too many damaged fibers. At the maximum Cᵥ, the expansion ratio (wet/dry) is also at its maximum. Again, beyond maximum Cᵥ, the Cᵥ decreases until the density of the tampon or plug reaches the molecular density of the base component (e.g., cellulose).

In contrast to the single plug disclosed in Steiger et al., the present invention provides a fluid-permeable bag containing a plurality of tablets of compressed, fibrous, absorbent material. These tablets comprise a fibrous mass of absorbent fibers compressed to a bulk density within about 20% of the maximum volume capacity. More preferably, the tablets are compressed to a bulk density between about 80% and 100% of the maximum volume capacity. As the 100% point is exceeded, the volume capacity decreases, and the absorption performance of the fibrous system decreases to levels incorporating less fiber and more force is required to produce the more dense tablets. Therefore, it is less economic to exceed 100% of the maximum volume capacity.

The maximum volume capacity provides good absorption and expansion for a defined tablet. For cellulosic fibers, the yield point (maximum Cᵥ) is reached at about 1.0 gm/cm³.

The fibrous, absorbent material includes bondable fibers, bondable fiber blends, and/or fibers combined with binding agents. This allows the compressed tablet to remain compressed. Preferably, at least a portion of the fibers are capable of hydrogen bonding. Hydrogen bonding holds the fibers in a compressed form until moisture breaks the bonds. Other bondable fibers may have a bondable surface treatment that is releasable in a moist (water vapor) or aqueous liquid environment. Binding agents may also be used to maintain the compression of the tablets, including without limitation, water-soluble binding agents, waxes, glues and the like.

Preferably fibers include, without limitation, cellulosic fibers and synthetic fibers such as polyesters, polyvinyl alcohols, polyolefins, polyamines, polyamides, polyacrylonitriles, and the like can also be used. A representative, non-limiting list of cellulosic fibers includes natural fibers such as cotton, wood pulp, jute, bagasse, silk, wool, and the like; and processed fibers such as regenerated cellulose, cellulose acetate, cellulose nitrate, rayon, and the like. Preferably, the cellulosic fibers are rayon or cotton, and more preferably, the fibers are rayon.

The fibers can also be multi-limbed, including multi-limbed regenerated cellulosic fibers and multi-limbed polyester or polyolefin fibers. A preferred source of multi-limbed regenerated cellulosic fibers are available as DANUFIL VY viscose rayon fibers from Acordis Ltd., Birmingham, England. These fibers are described in detail in Wilkes et al., U.S. Pat. No. 5,458,835, the disclosure of which is hereby incorporated by reference.

It is expected that any multi-limbed commercial fiber or even other such fibers not currently commercially available, would be useful in the practice of this invention.

Again, additional fibers may be added. These additional fibers may include synthetic fibers such as polyesters, polyvinyl alcohols, polyolefins, polyamines, polyamides, polyacrylonitriles, and the like.

Different fibers can withstand varying levels of compression before exhibiting significant levels structural damage. We have found that the multi-limbed rayon fibers, for example, encounter less damage upon high compression than other fibers such as cotton or polyester (PET) fibers. This type of fiber can better use the region of the maximum volume capacity and thus can be used to optimize tampon characteristics.

The tablets may be formed of 100% of a single fiber type, or they may be formed of a blend of two or more different fibers. For example, blends of multi-limbed and non-limbed rayon may be used. Additionally, blends of rayon and one or more of the fibers listed above can be used.

If a blend of fibers is used, they are preferably blended to a substantially uniform mixture of fibers. Those of ordinary skill in the art know useful fiber blending operations. For example, the fibers can be continuously metered into a saw-tooth opener. The blended fibers can be transported, e.g., by air through a conduit to a carding station to form a fibrous web. The fibrous web is preferably calendered to impart a minor amount of compression. To form a tablet, the web can be formed into a yarn that is then fed into a compression unit working similarly to a rotary tablet compressing/manufacturing unit.

The fluid input rate and the total expansion, retention and absorption capacity of the bag-type tampon is increased by forming a bag containing at least two independent pieces of fiber plugs that have been compressed to a bulk density which is within 20% of the maximum Cᵥ. Preferably, the fiber is compressed to at least about 0.5 g/cm³, and more preferably, at least about 0.6 g/cm³. Most preferably, the tablets have a bulk density of at least about 0.8 g/cm³, and a particularly preferred embodiment comprises regenerated cellulose fibers, such as rayon fibers, and has a bulk density of about 0.8 to about 1.2 g/cm³. By having a higher density, the overall volume capacity is increased and by having at least two pieces of absorbent compressed fiber, the fluid intake rate is higher. The number of pieces in the tampon can vary from two to 500.

In a preferred embodiment, a bag-type tampon is filled with substantially cylindrical tablets sized 3 mm in diameter by 5 mm long made from a blend of 75% DANUFIL VY rayon and 25% DANUFIL V rayon compressed to a density of 0.9 g/cm³. Each bag contains between 80 and 120 tablets to give total tablet weight of 4.5 g.

The tablets are compressed by taking 0.04 g of fiber blend, placing it into a 3 mm diameter chamber and compressing it with a 3 mm diameter piston fitted onto a hydraulic press.

The overwrap or bag-forming material may be any fluid-permeable material that is capable of containing the tablets and any other associated material within the bag. Suitable bag materials include those with open mesh structures such as woven, nonwoven, and knit textiles; apertured films; polymeric nets; and the like. Preferably, the fluid-permeable materials are soft, flexible, and have small apertures therethrough. Additional desirable features can include biodegradability.

Useful bag materials enable easy bag formation and sealing. Therefore, qualities such as thermobondability, high tensile strength, high masking effect to prevent users from noticing the tablets and softness are desirable.

It is not necessary for the overwrap material to have noticeable apertures therein, but some materials having noticeable apertures have been used satisfactorily. The apertures must, however be small enough to keep small pieces and/or fibers from escaping through the overwrap and to prevent edges or corners of pieces from protruding through the overwrap. Protrusion of pieces through apertures may interfere with ejection of absorbent article or tampon from applicator. Thus, the outer surface of the overwrap should be as smooth and have as low a coefficient of friction as possible. This provides at least two benefits: (1) the force required to eject the tampon is reduced, and (2) it reduces the damage otherwise caused by scraping of soft, tender tissue within the vagina during insertion, wearing and removal.

The overwrap must be strong enough to prevent rupturing during handling, insertion, removal and from vaginal pressures during use.

Additionally, the overwrap used for a tampon should provide means to remove the bag after use, e.g., an extension of the bag, itself, or an attached element such as a removal string. Examples of materials suitable for use as removal string include cotton string and any string sufficiently strong enough to withstand removal forces used to removal the tampon from the body cavity. Polyester strings may also be used.

These tablets can be used in baby diapers, sanitary napkins, pantiliners, interlabial devices, wipes, or in any article that requires absorbency, retention and expansion.

The present invention will be further understood by reference to the following specific Examples that are illustrative of the composition, form and method of producing the absorbent article of the present invention. It is to be understood that many variations of composition, form and method of producing the absorbent article would be apparent to those skilled in the art. The following Examples, wherein parts and percentages are by weight unless otherwise indicated, are only illustrative.

### EXAMPLE 1

A series of fibrous webs were formed by adding a measured amount of staple length fibers having the compositions (by wt-%) identified in Table 1 below. For each web, the fibers were intimately mixed in and carded to form the fibrous web. The web was then compressed to form a plug. The fibers used in these examples include HYDROCEL, a treated rayon fiber providing carboxymethyl cellulose on at least the surface thereof, available from Acordis Ltd., Birmingham, England; DANUFIL VY, multi-limbed viscose rayon fibers from Acordis Ltd.; DANUFIL V, standard viscose rayon fibers from Acordis Ltd.; cotton fibers; and 1.5 denier polyethylene terephthalate (“PET”) fibers, T-121 PET from KoSa, Houston, Tex., USA.

TABLE 1

| | HYDRO- | DANUFIL | DANUFIL | | 1.5 denier | Wood |
|---|---|---|---|---|---|---|
| Ex. | CEL | VY | V | Cotton | PET T-121 | Pulp |
| I | 50 | 50 | | | | |
| II | | 70 | | 30 | | |
| III | | 80 | | | 20 | |
| IV | | 75 | 25 | | | |
| V | | | | | | 100 |

Table 2 shows the plug composition, density and weight capacity.

TABLE 2

| | | | Volume | Compress. |
|---|---|---|---|---|
| | Density | Weight Capacity | Capacity | Pressure |
| Sample | (g/cm³) | (g fluid/g fiber) | (ml/cm³⁾ | (psi) |
| I | 0.28 | 7.16 | 2.01 | 2500 |
| I | 0.30 | 6.67 | 2.00 | 2500 |
| I | 0.39 | 7.05 | 2.74 | 6400 |
| I | 0.42 | 6.84 | 2.84 | 6400 |
| I | 0.50 | 6.74 | 3.40 | 10000 |
| II | 0.43 | 3.63 | 1.55 | 5100 |
| II | 0.44 | 3.49 | 1.54 | 5100 |
| II | 0.59 | 3.60 | 2.13 | 10000 |
| II | 0.63 | 3.62 | 2.28 | 10000 |
| II | 0.73 | 3.21 | 2.33 | 14000 |
| II | 0.76 | 3.15 | 2.39 | 14000 |
| III | 0.41 | 4.34 | 1.80 | 7600 |
| III | 0.42 | 4.3 | 1.80 | 7600 |
| III | 0.52 | 4.14 | 2.15 | 10000 |
| III | 0.63 | 3.99 | 2.51 | 14000 |
| IV | 0.27 | 5.29 | 1.44 | 2500 |
| IV | 0.28 | 5.01 | 1.39 | 2500 |
| IV | 0.43 | 5.07 | 2.19 | 5100 |
| IV | 0.48 | 4.84 | 2.33 | 5100 |
| IV | 0.62 | 4.33 | 2.68 | 10000 |
| IV | 0.67 | 4.46 | 3.00 | 10000 |
| IV | 0.75 | 4.07 | 3.04 | 14000 |
| IV | 0.79 | 3.88 | 3.06 | 14000 |
| V* | 0.1 | 4.03 | 0.40 | |
| V* | 0.2 | 3.64 | 0.73 | |
| V* | 0.3 | 3.24 | 0.97 | |
| V* | 0.4 | 2.85 | 1.14 | |
| V* | 0.5 | 2.46 | 1.23 | |

*The data provided for Sample V was extrapolated from previous testing. The reported data represent prophetic data; they are not actual data points measured from wood pulp plugs.

Although maximum C_{V} was not reached due to limitations in press equipment, it can be extrapolated from these data and the teaching of Steiger et al. The extrapolated data provide the maximum Cᵥ for each example as shown in Table 2a, below.

TABLE 2a

| | | | | Density @ |
|---|---|---|---|---|
| | | Density @ | 80% of | 80% |
| | Maximum Cᵥ | Max. Cᵥ | Max. Cᵥ | Max. Cᵥ |
| Sample | (ml/cm³) | (g/cm³) | (ml/cm³) | (g/cm³) |
| I | | | | |
| II | 3.11 | 1.4-1.53 | 2.51 | 0.82 |
| III | 3.92 | 1.54-1.61 | 3.14 | 0.87 |
| IV | | | | |
| V | 1.25 | 0.55-0.57 | 1.0 | 0.32 |

Thus, the data illustrate that cellulosic systems can approach their maximum Cᵥ at densities as low as 0.5 g/cm³, while other systems may approach their maximum Cᵥ at densities about 1.5 g/cm³. 80% of the maximum Cᵥ for these latter systems occurs at densities of about 0.8 g/cm³.

### EXAMPLE 2

Bag-type tampons were prepared with the following specifications: A mixture of 75 wt-% DANUFIL VY multi-limbed rayon fibers and 25 wt-% DANUFIL V rayon fibers was processed as above but formed into a yarn instead of being formed into a plug. The yarn was compressed under 1685 PSI and formed into tablets. Each tablet weighed between 0.04 to 0.05 g, was approximately 3.2 mm (diameter) by 7 mm (length), and had a density of approximately 0.9 g/cc. Between 90 and 112 tablets were placed in a bag made from ENKA 4128, bicomponent (polyethylene over polyester) fibers available from PGI Nonwovens, Dayton, N.J., USA. The bag weighed 0.92 g. The finished product was placed into a standard applicator.

All tampons in this test were measured for absorbency by the Syngina Test. This test is described in Federal Register, Part III, Department of Health and Human Services, Food and Drug Administration (21 CFR Part 801, pp. 37263-4, Sep. 23, 1988). Measurements of the tampons were taken both prior to testing, during testing and after testing. The testing was run in duplicate. Table 3 represents the first test results; Table 4 represent the second test results.

TABLE 3

| | Initial | | | Wet Size |
|---|---|---|---|---|
| | Size | | Wet Size | after SO |
| | (Dia. × | Absor- | (Dia. × | (Dia. × |
| | length) | bency | length) | length) |
| Sample # | (mm) | (ml) | (mm) | (mm) |
| Bag-type 1 | 13 × 60 | 14.96 | 35 × 65 | 35 × 65 |
| Bag-type 2 | 13 × 60 | 15.35 | 35 × 70 | 35 × 70 |
| Commercial | 12 × 55 | 8.63 | 17 × 55 | 15 × 55 |
| Regular 1 | | | | |
| Commercial | 12 × 55 | 8.90 | 16 × 55 | 15 × 55 |
| Regular 2 | | | | |
| Commercial | 13 × 55 | 11.16 | 20 × 56 | 18 × 56 |
| Super 1 | | | | |
| Commercial | 13 × 55 | 9.85 | 18 × 55 | 18 × 55 |
| Super 2 | | | | |
| Commercial | 15 × 55 | 13.73 | 21 × 55 | 22 × 58 |
| Super Plus 1 | | | | |
| Commercial | 15 × 55 | 12.94 | 21 × 58 | 22 × 58 |
| Super Plus 2 | | | | |

TABLE 4

| | Initial | | | Wet Size |
|---|---|---|---|---|
| | Size | | Wet Size | after SO** |
| | (Dia. × | Absor- | (Dia. × | (Dia. × |
| | length) | bency | length) | length) |
| Sample # | (mm) | (ml) | (mm) | (mm) |
| Bag-type 1 | 15 × 60 | 16.98 | 32 × 70 | 35 × 70 |
| Bag-type 2 | 15 × 62 | 16.20 | 35 × 70 | 35 × 70 |
| Commercial | 14 × 54 | 9.44 | 17 × 55 | 15 × 55 |
| Regular 1 | | | | |
| Commercial | 13 × 54 | 9.70 | 16 × 55 | 17 × 55 |
| Regular 2 | | | | |
| Commercial | 15 × 55 | 11.93 | 18 × 55 | 18 × 55 |
| Super 1 | | | | |
| Commercial | 15 × 55 | 12.30 | 19 × 55 | 19 × 55 |
| Super 2 | | | | |
| Commercial | 17 × 55 | 15.45 | 23 × 55 | 22 × 55 |
| Super Plus 1 | | | | |
| Commercial | 17 × 55 | 15.80 | 22 × 55 | 20 × 55 |
| Super Plus 2 | | | | |
| **Squeeze-Out or fluid retention test. Samples are placed in a dry syngina apparatus described in 21 CFR Part 801 (see above) after having been tested for absorbency. The pressure of the dry syngina apparatus is increased until the product starts letting fluid out. The pressure at which fluid is first “squeezed-out” is recorded. The density is then increased to a maximum of 100 inches of water. That pressure is held until the tampon stops dripping fluid out. The weight of the | | | | |
| # tampon is then recorded to see how much fluid was held inside it. | | | | |

The diameter of the bag-type tampon shows nearly a two-fold increase when wet, and the same trend is observed even after squeeze-out. Thus, the absorbent articles of the present invention show a substantial increase in expansion capacity over conventional tampons.

The specification and examples above are presented to aid in the complete and non-limiting understanding of the invention disclosed herein. Since many variations and embodiments of the invention can be made without departing from its spirit and scope, the invention resides in the claims hereinafter appended.

What is claimed is:

1. An absorbent article comprising a fluid-permeable overwrap containing a plurality of tablets of compressed, fibrous, absorbent material having a bulk density within about 20% of said absorbent material's maximum volume capacity.

2. An absorbent article of claim 1, wherein the fibrous, absorbent material comprises a binding agent.

3. An absorbent article of claim 2, wherein the binding agent is water soluble.

4. An absorbent article of claim 1, wherein the fibrous, absorbent material comprises a bondable fiber.

5. An absorbent article of claim 4, wherein the bondable fiber is hydrogen bondable.

6. An absorbent article of claim 4, wherein said bondable fiber comprises cellulosic fibers.

7. An absorbent article of claim 6, wherein said cellulosic fibers comprise multi-limbed regenerated cellulosic fibers.

8. An absorbent article of claim 6, wherein said cellulosic fibers comprise non-limbed cellulosic fibers.

9. An absorbent article of claim 6, wherein said cellulosic fibers comprise a mixture of multi-limbed regenerated cellulosic fiber and non-limbed regenerated cellulosic fiber.

10. An absorbent article of claim 6, wherein said cellulosic fibers comprise a mixture of about 60 wt-% to about 100 wt-% of multi-limbed regenerated cellulosic fibers and about 0 wt-% to about 40 wt-% of non-limbed cellulosic regenerated fibers.

11. The absorbent article of claim 10, wherein said mixture is about 70 wt-% to about 80 wt-% multi-limbed regenerated cellulosic fiber and about 30 wt-% to about 20 wt-% non-limbed regenerated cellulosic fiber.

12. An absorbent article of claim 1, wherein said tablet has a predetermined density in the range of about 0.8 to 1.2 gm/cc.

13. An absorbent article of claim 1, wherein said overwrap comprises a nonwoven material.

14. An absorbent article of claim 1, wherein said overwrap comprises an apertured film.

15. An absorbent article of claim 1, wherein said overwrap is liquid-permeable.

16. An absorbent article of claim 1, wherein said overwrap comprises a bag.

17. An absorbent article of claim 1 comprising between two and 500 tablets of absorbent material.

18. An absorbent article of claim 17 comprising about 55 to about 60 tablets of absorbent material.

19. An absorbent article of claim 1, wherein said absorbent article is a tampon.

20. An absorbent article comprising a liquid-permeable bag containing a plurality of tablets of compressed, fibrous, absorbent material, said tablets having a bulk density of at least about 0.5 g/cm³.

21. The absorbent article of claim 20 wherein the tablets have a bulk density of at least about 0.8 g/cm³.

22. The absorbent article of claim 21 wherein the tablets have a bulk density of about 0.8 to about 1.2 g/cm³.

23. The absorbent article of claim 20, wherein said fibrous, absorbent material comprises a mixture of multi-limbed regenerated cellulosic fibers and non-limbed regenerated fibers.

24. An absorbent article of claim 23, wherein said absorbent material comprises a mixture of about 60 wt-% to about 100 wt-% of multi-limbed regenerated cellulosic fibers and about 0 wt-% to about 40 wt-% of non-limbed cellulosic regenerated fibers.

25. An absorbent article of claim 24, wherein said absorbent material comprises a mixture of about 70 wt-% to about 80 wt-% of multi-limbed regenerated cellulosic fibers and about 30 wt-% to about 20 wt-% of non-limbed cellulosic regenerated fibers.

26. A method of making an absorbent article, said method comprising the steps of:
a) mixing fibrous, absorbent material;
b) forming the mixed fibrous, absorbent material into compressed tablets;
c) placing the compressed tablets into an overwrap; and
d) sealing the overwrap.

27. A method of claim 26, wherein the absorbent material is compressed to form tablets having a predetermined density within 20% of said absorbent material's maximum volume capacity.

28. A method of claim 26, further comprising placing the sealed overwrap into a tampon applicator.

29. A method of claim 26, wherein the overwrap comprises a bag.

30. A method of claim 26, wherein the overwrap comprises a nonwoven material.

31. A method of claim 26, wherein the step of compressing the tablets comprises compressing the tablets to a bulk density of at least about 0.5 g/cm³.

32. A method of claim 31, wherein the step of compressing the tablets comprises compressing the tablets to a bulk density of at least about 0.8 g/cm³.

33. A method of claim 32, wherein the step of compressing the tablets comprises compressing the tablets to a bulk density of about 0.8 g/cm³ to about 1.2 g/cm³.

34. A method of claim 29, further comprising the step of attaching a withdrawal string to the bag.

35. A method of claim 34, further comprising the step of placing the absorbent article into a tampon applicator.

36. A method according to claim 26, wherein the step of sealing the overwrap comprises sealing the overwrap to a barrier material to form a pad.
